(19) United States
(12) Patent Application Publication
(10) Pub. No.: US 2022/0308568 A1
(43) Pub. Date: Sep. 29, 2022

(54) SYSTEM AND METHOD FOR MONITORING SOIL GAS AND PERFORMING RESPONSIVE PROCESSING ON BASIS OF RESULT OF MONITORING

(71) Applicant: Korea University Research and Business Foundation, Seoul (KR)

(72) Inventors: Yun Yeong OH, Busan (KR); Seong Taek YUN, Seoul (KR)

(73) Assignee: Korea University Research and Business Foundation, Seoul (KR)

(21) Appl. No.: 17/294,028

(22) PCT Filed: Oct. 17, 2019
(86) PCT No.: PCT/KR2019/013638

§ 371 Date: Oct. 4, 2021

(30) Foreign Application Priority Data
Nov. 16, 2018 (KR) 10-2018-0141240

(51) Int. Cl. G05B 23/02

(52) U.S. Cl. CPC G05B 23/027; G05B 23/0221; G05B 23/0235; G05B 23/0272

(57) ABSTRACT

The present invention relates to an universal integrated environmental monitoring and management technique for operating underground storage sites of gaseous substances including CO₂ capture and storage (CCS) site. According to the present invention, Firstly, a base dataset for observed soil gases and related surrounding environmental variables by a step of configuring/refining procedures. Next, extracting the time varying characteristics of the base dataset using wavelet-based multiresolution state-space modeling, and identifying the driving forces that governing the soil gases dynamics and evaluating their contributions through multiscale time-frequency domain correlation analysis. And finally, predicting and forecasting future scenarios with deep leaning models which intensively trained by the key driving forces. Furthermore, the present invention can provide quantitative based for analyzing the causation between driving forces and observed soil gases. In addition, the present invention can effectively be used to detect early leakage signs and to assess environmental impacts of leakage based on the identification, evaluation, and prediction results.

### TECHNICAL FIELD

The present invention relates to an integrated environmental monitoring and management technique. More particularly, the present invention relates to the integrated method for quantitatively identifying, evaluating, and predicting the natural background variation (i.e., baseline) of soil gases (CO₂, NO₂, CH₄, CO, C₂H₆, N₂O, SO₂, Rn) and environmental factors (i.e., driving forces) that governing their baseline at underground storage sites for gaseous substances including carbon dioxide capture and storage (CCS) using real-time monitoring data. In addition, the method includes detecting early sing of leakage and evaluating and predicting the environmental impact of leaks on surroundings based on the identification, evaluation, and prediction results.

### BACKGROUND ART

After the Paris Climate Agreement held in November 2015, countries around the world has set targets for CO₂ reduction to reduce the load of anthropogenic carbon dioxide (CO₂) emitted into the atmosphere. Moreover, various research and projects related to the carbon dioxide capture and storage (CCS) also have been carried out as an active countermeasure to reduce CO₂ load. Especially in Norway and Australia, the commercial storage facilities has already been installed and actively operated, and these facilities are expected to increase in the future.

In South Korea, Pilot scale near subsurface CO₂ injection tests and research projects for establishing groundwork for the integrated CCS environmental management system has been conducted during 2014-2020. In addition, to achieve Korean CO2 reduction target for 2030, large scale R&D and demonstration projects are also being carried out to secure massive offshore CCS sites.

CO₂ stored in underground could release to groundsurface due to the natural and/or anthropogenic factors. This release process may be interpreted as leakage. This leakage inevitably has physical, chemical, and biological impacts on both subsurface and groundsurface environment and ecosystem. Therefore, when designing an injection and storage facility, environmental impact assessment and prediction for the potential leakage should be prepared.

In addition, to secure the safety of the CCS site, integrated environmental monitoring and management system need to be operated for a long time even after injection is completed. Since CO₂ leaking from an underground reservoir eventually reaches as gaseous phase at the near-surface, CO₂ concentration and flux at the near-surface can be used to the direct indicators for detecting early sign of CO₂ leakage.

However, the concentration and flux CO₂ in near-surface are very complex in time and spaces regardless of leakage due to the complex interactions among the atmosphere-soil and biological systems. Therefore, it is very difficult to detect the early sign of leakage using near-surface soil gas monitoring.

Besides, the real-time observed soil gases (CO₂, NO₂, CH₄, CO, C₂H₆, N₂O, SO₂, Rn) data and accompanying environmental variables (hydro-meteorological data, soil physicochemical properties, influence of plants, etc.) are generally considered as fixed values based on the i.i.d (independent and identically distributed) assumption in the conventional soil gas monitoring and analysis techniques. Accordingly, the variability of soil gases data and accompanying environmental variables with inherent nonstationary and interdependence could not been properly interpreted.

In particular, the concentration and flux of near-surface soil gases are also greatly affected by change in diurnal and seasonal cycles of air-soil temperature, surface atmospheric turbulent flow, and soil respiration.

Accordingly, even though the resolution of sensor is dramatically increased, the fixed value-based conventional interpretation methods could not be restricted to interpret the observed results only. Therefore, it is necessary to develop the integrated method that could quantify both observed data and their factors (i.e., driving forces) behind them in real-time.

### DISCLOSURE

### Technical Problem

The present invention has been made to solve above mentioned problems, and it is one object of the present invention to reflect the time varying (i.e., dynamic) characteristics of various environmental factors that governing the dynamics of soil gases (CO₂, NO₂, CH₄, CO, C₂H₆, N₂O, SO₂, Rn) and additional environmental variables incidentally observed with them.

It is another object of the present invention to systematically separate and identify key environmental driving forces that cause change in observation values and the effects thereof using multi-resolution time-frequency domain analysis.

It is still another object of the present invention to quantitatively evaluate environmental driving forces and contribution thereof.

It is still another object of the present invention to configure a deep neural network using observed values and environmental factors at the same time.

It is still another object of the present invention to present a quantitative basis for the causal interpretation between dynamic characteristic values and predicted values for optimal learning in configuring a deep neural network.

It is still another object of the present invention to intensively learn the key spatiotemporal change characteristics of target soil gases and environmental influencing factors.

It is still another object of the present invention to efficiently perform precise prediction.

It is still another object of the present invention to generally use for real-time measurement data of various gaseous substances, such as concentration, flux, and isotope ratio, without being limited to specific types of soil gases.

It is still another object of the present invention to detect leakage signals early by effectively separating/identifying natural background variation and accidental (anthropogenic) leakage.

It is yet another object of the present invention to contribute to the vulnerability and/or risk assessment in target underground storage sites and surroundings that influenced by specific environmental factors and/or substances.

### Technical Solution

In accordance with one aspect of the present invention, provided is a method of operating a soil gas monitoring and response system, the method including a step of configuring a base dataset for observation data of soil gases and related environments; a step of identifying and extracting dynamic characteristics of the configured base dataset; and a step of identifying and evaluating a driving force for soil gases based on the extracted dynamic characteristics, wherein an optimal response scenario is provided based on the identified and evaluated driving force.

According to one embodiment, the step of configuring a base dataset for observation data of soil gases and related environments may include a step of configuring and aligning, as the base dataset, a data matrix according to observation items and observation time resolution of a complex environmental measurement dataset including the soil gases; a step of performing interpolation processing on missing data for each temporal domain resolution or temporal observation interval for the sorted base dataset; and a step of performing noise filtering on data of the interpolated base dataset, and standardizing and normalizing the noise-filtered results.

According to one embodiment, the step of identifying and extracting dynamic characteristics of the configured base dataset may include a step of performing state-space modeling on the configured base dataset for each temporal domain resolution; a step of selecting an optimal state-space model according to the temporal domain resolution of the configured base dataset; a step of selecting a potential driving force group of the selected optimal state-space model; a step of extracting time-dependent variation characteristics of the selected potential driving force group; and a step of quantifying dynamic characteristics of a main time-frequency domain through Wavelet analysis for the extracted variation characteristics. In this case, the variation characteristics may include at least one of time-dependent dynamic characteristics, time-varying characteristics, spatial characteristics, and spatiotemporal characteristics of the selected potential driving force group.

According to one embodiment, the step of selecting an optimal state-space model may include a step of selecting the number of optimal potential driving forces and a form of a residual covariance matrix based on model diagnostic indexes (AIC/AICc/BIC) and explanatory power (loading).

According to one embodiment, the step of identifying and evaluating driving forces for soil gases based on the extracted dynamic characteristics may include a step of diagnosing multi-resolution correlation between observation data and potential driving forces of an optimal state-space model selected according to a temporal domain resolution of the configured base dataset, and performing correlation diagnosis reflecting time delay and phase change between the potential driving forces and the observation data; a step of selecting a highest correlation scale between the potential driving forces and the observation data based on results of the performed correlation diagnosis; a step of identifying a driving force using a Wavelet energy ratio between the potential driving forces and the observation data and a correlation of the selected highest correlation scale; and a step of evaluating relative contribution by processing a linear combination between a cumulative energy ratio of the selected highest correlation scale and an explanatory power index of the state-space model.

According to one embodiment, the method of operating a soil gas monitoring and response system may further include a step of constructing a deep learning model for real-time diagnosis of the driving force.

According to one embodiment, the step of constructing a deep learning model may include a step of constructing, as a deep neural network model, a deep learning model using the observation data of soil gases and related environments and the identified and evaluated driving force as input data; a step of quantifying a training indicator by selecting the training indicator based on multi-resolution dynamic characteristics of the observation data of soil gases and related environments and the identified and evaluated driving force; a step of optimizing a prediction model based on residual verification of observation values measured from the observation data of soil gases and related environments and prediction values that predicted using the deep learning model and multi-resolution analysis of residuals; and a step of generating a tuned pre-trained network group by performing optimization processing by key environmental forces.

According to one embodiment, the method of operating a soil gas monitoring and response system may further include a step of constructing a real-time response system for providing the optimal response scenarios.

According to one embodiment, the step of constructing a real-time response system may include a step of constructing a real-time diagnosis system using the generated tuned pre-trained network group; a step of calculating a permissible range of a natural background variation (i.e., baseline) by selecting a threshold value of the baseline; a step of re-identifying a driving force for data determined as an outlier with respect to the threshold value, and reconfiguring an optimized deep training network group based on the re-identified driving force; a step of identifying a driving force according to prediction results of the outlier, evaluating relative contribution, and selecting an alarm priority; a step of generating a real-time change and response scenario for each cause of the outlier; and a step of generating an alarm signal according to the generated a real-time change and response scenario, and providing an optimal response scenario.

In accordance with another aspect of the present invention, provided is a soil gas monitoring and response system including a preprocessor for configuring a base dataset for observation data of soil gases and related environments; a dynamic characteristic processor for identifying and extracting dynamic characteristics of the configured base dataset; and a driving force processor for identifying and evaluating a driving force for soil gases based on the extracted dynamic characteristics, wherein an optimal response scenario is provided based on the identified and evaluated driving force.

According to one embodiment, the preprocessor may configure and align, as the base dataset, a data matrix according to observation items and observation time resolution of a complex environmental measurement dataset including the soil gases; may perform interpolation processing on missing data for each temporal domain resolution or temporal observation interval for the sorted base dataset; and may perform noise filtering on data of the interpolated base dataset, and standardize and normalize the noise-filtered results.

According to one embodiment, the dynamic characteristic processor may perform state-space modeling on the configured base dataset for each temporal domain resolution; may select an optimal state-space model according to the temporal domain resolution of the configured base dataset; may select a potential driving force group of the selected optimal state-space model; may extract time-dependent variation characteristics of the selected potential driving force group; and may quantify dynamic characteristics of a main time-frequency domain through Wavelet analysis for the extracted variation characteristics.

According to one embodiment, the driving force processor may diagnose multi-resolution correlation between observation data and potential driving forces of an optimal state-space model selected according to a temporal domain resolution of the configured base dataset, and may perform correlation diagnosis reflecting time delay and phase change between the potential driving forces and the observation data; may select a highest correlation time-frequency scale between the potential driving forces and the observation data based on results of the performed correlation diagnosis; may identify a driving force using a Wavelet energy ratio between the potential driving forces and the observation data and a correlation of the selected highest correlation scale; and may evaluate relative contribution by processing a linear combination between a cumulative energy ratio of the selected highest correlation scale and an explanatory power index (loading) of the state-space model.

### Advantageous Effects

According to one embodiment, the time varying structural characteristics of various environmental factors that governing the dynamics of various soil gases (CO₂, NO₂, CH₄, CO, C₂H₆, N₂O, SO₂, Rn) and other environmental observation data (hydro-meteorological data, soil physical/chemical property data, biological respiration data) also can be reflected.

According to one embodiment, key environmental driving forces that cause change in observation values and the effects thereof can be systematically separated and identified using multi-resolution time-frequency domain analysis.

According to one embodiment, environmental driving forces and contribution thereof can be quantitatively evaluated.

According to one embodiment, a deep neural network can be configured using observed values and environmental factors as input at the same time.

According to one embodiment, by configuring a deep neural network, a quantitative basis for the causality of a characteristic value and prediction for optimal learning (training) can be presented.

According to one embodiment, the key spatiotemporal characteristics of target soil gases and environmental influencing factors can be intensively learned (trained).

According to one embodiment, precise prediction can be efficiently performed.

According to one embodiment, real-time measurement data of various gaseous substances can be universally used without being limited to specific types of soil gases.

According to one embodiment, a leakage signal can be detected early through by effectively separating/identifying the natural background variation (i.e., baseline) and accidental (anthropogenic) leakage signs.

According to one embodiment, the present invention can contribute to improve the efficiency of the vulnerability and risk assessment for target underground storage sites and surrounding environments that influenced by specific environmental factors and/or substances.

### DESCRIPTION OF DRAWINGS

FIG. 1 is a block diagram for explaining an entire system according to one embodiment.

FIG. 2 is a flowchart for explaining a method of operating an entire system according to one embodiment.

FIG. 3 is a flowchart for explaining a method of configuring a base dataset for observation data of soil gases and related environments.

FIG. 4 is a flowchart for explaining a process of identifying and extracting the dynamic characteristics of a configured base dataset.

FIG. 5 is a flowchart for explaining a process of identifying and evaluating driving forces for soil gases based on extracted dynamic characteristics.

FIG. 6 is a flowchart for explaining a process of constructing a deep learning neural network model.

FIG. 7 is a flowchart for explaining a process of constructing a real-time response system.

FIG. 8 shows a test site divided into zones for an experiment to observe emission of CO₂.

FIG. 9A shows a Wavelet energy distribution, and shows the distribution of Wavelet energy (Ew) for each frequency for raw data of observation variables and Wavelet de-noising (WD) data by discrete Wavelet transform (DWT).

FIG. 9B includes graphs showing raw data and Wavelet de-noising (WD) data of major observation data.

FIG. 10 shows potential driving force groups (Potential EDs; potential environmental drivers=potential driving force groups) extracted by state-space modeling.

FIG. 11A shows a multi-resolution scale-versus-correlation diagram for confirming the core frequency (i.e. among detailed components by the discrete Wavelet transform of observation data.

FIG. 11B shows a scale-versus-correlation diagram for both approximate components and detailed components.

FIG. 12A shows an example of deep learning prediction using only observation data.

FIG. 12B is a diagram showing a result of reinforcing learning using observation values and identified key features in FIG. 12A.

### BEST MODE

Specific structural and functional descriptions of embodiments according to the concept of the present invention disclosed herein are merely illustrative for the purpose of explaining the embodiments according to the concept of the present invention. Furthermore, the embodiments according to the concept of the present invention can be implemented in various forms and the present invention is not limited to the embodiments described herein.

The embodiments according to the concept of the present invention may be implemented in various forms as various modifications may be made. The embodiments will be described in detail herein with reference to the drawings. However, it should be understood that the present invention is not limited to the embodiments according to the concept of the present invention, but includes changes, equivalents, or alternatives falling within the spirit and scope of the present invention.

The terms such as “first” and “second” are used herein merely to describe a variety of constituent elements, but the constituent elements are not limited by the terms. The terms are used only for the purpose of distinguishing one constituent element from another constituent element. For example, a first element may be termed a second element and a second element may be termed a first element without departing from the teachings of the present invention.

It should be understood that when an element is referred to as being “connected to” or “coupled to” another element, the element may be directly connected or coupled to the other element or intervening elements may be present. In contrast, when an element is referred to as being “directly connected to” or “directly coupled to” another element, there are no intervening elements present. Other words used to describe the relationship between elements or layers should be interpreted in a like fashion (e.g., “between,” versus “directly between,” “adjacent,” versus “directly adjacent,” etc.).

The terms used in the present specification are used to explain a specific exemplary embodiment and not to limit the present inventive concept. Thus, the expression of singularity in the present specification includes the expression of plurality unless clearly specified otherwise in context. Also, terms such as “include” or “comprise” should be construed as denoting that a certain characteristic, number, step, operation, constituent element, component or a combination thereof exists and not as excluding the existence of or a possibility of an addition of one or more other characteristics, numbers, steps, operations, constituent elements, components or combinations thereof.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, preferred embodiments of the present invention will be described in detail with reference to the accompanying drawings. However, the scope of the present invention is not limited by these embodiments. Like drawing symbols in the drawings denote like elements.

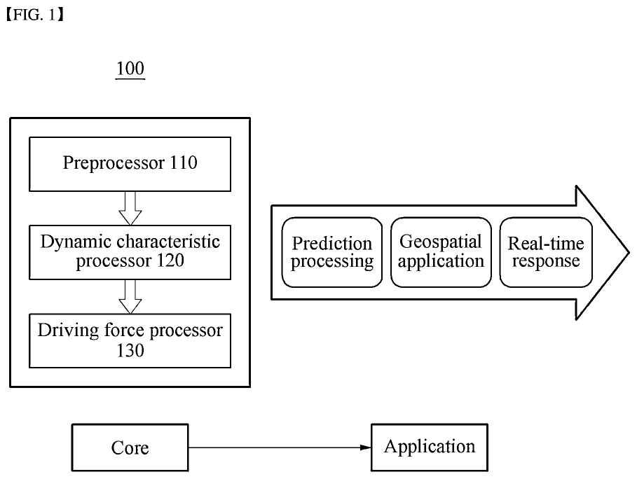

FIG. 1 is a block diagram for explaining a soil gas monitoring and response system 100 according to one embodiment.

The soil gas monitoring and response system 100 according to one embodiment performs operation of quantitatively identifying, evaluating, and predicting the natural background variation (i.e., baseline) of soil gases (CO₂, NO₂, CH₄, CO, C₂H₆, N₂O, SO₂, Rn) and environmental factors (i.e., driving forces) that governing their baseline at underground storage sites for gaseous substances including carbon dioxide capture and storage (CCS) using real-time monitoring data.

When the soil gas monitoring and response system 100 is used, integrated environmental monitoring of soil gases and management based on the monitoring may be provided, and the structural characteristics and causality of soil gases (CO₂, NO₂, CH₄, CO, C₂H₆, N₂O, SO₂, Rn) and environmental observation data (hydro-meteorological data, soil physical/chemical property data, biological respiration data) may be secured through the dynamic factor analysis.

For reference, conventional factor analysis corresponds to a quantification technique for major factors through dimensionality reduction of multivariate data. In addition, dynamic factor analysis may extract and analyze common factors inherent in observational data through combination of time series analysis and factor analysis. For reference, time series analysis is a generic term for stochastic analysis techniques for data that are continuously observed in time order. In addition, the soil gas monitoring and response system 100 according to one embodiment may specifically identify environmental driving forces and quantitatively evaluate influence by quantifying the similarity between dynamic factors and the time-frequency domain of observation data through Wavelet-based multi-resolution correlation analysis.

Wavelet analysis, as used in this specification, may be interpreted as a multi-resolution analysis technique in the time-frequency domain using Wavelet transformation of time series data. Wavelet analysis is specialized in decomposing observation data into various time-frequency domains. That is, Wavelet analysis is a method of analyzing a time domain and a frequency domain at the same time, and may be applied to both continuous and discrete signals, and may be widely applied to such as fault diagnosis.

Fast Fourier transform (FFT) has a disadvantage in that information in a time interval is lost because information in measurement data is averaged over time. Accordingly, Wavelet transform is particularly useful for analysis of non-stationary signals or transient signals in which defect frequency changes with time. Classical short-time Fourier transform (STFT) or Gabor transform uses a fixed filter window function of a new size to replace problems limited to a single frequency band. On the other hand, Wavelet transform variably uses a narrow window function in a high frequency band and a wide window function in a low frequency band. Accordingly, Wavelet transform is also called constant relative bandwidth analysis, and has a characteristic that the change width of a frequency band is always proportional to a frequency value.

The soil gas monitoring and response system 100 according to one embodiment performs monitoring in consideration of changes occurring in time series for each numerical value constituting soil gases based on Wavelet analysis. Accordingly, the soil gas monitoring and response system 100 may analyze a time domain and a frequency domain at the same time, and may monitor both continuous and discrete signals.

In addition, the soil gas monitoring and response system 100 according to one embodiment may more intensively learn the key spatiotemporal characteristics of a soil gas (e.g., CO₂) by using observation data of soil gases and related environments and environmental factors as input data at the same time through deep learning, thereby continuously improving prediction of observation values and factors.

That is, the soil gas monitoring and response system 100 according to one embodiment may intensively learn (focused training) the key spatiotemporal characteristics of the natural background variation of soil gases by constructing a deep learning model that uses observation data of soil gases and related environmental factors as input data at the same time, thereby continuously improving prediction of observation values and factors.

Spatiotemporal data mining by the soil gas monitoring and response system 100 corresponds to a data-driven big data analysis technique that extracts and analyzes the major characteristic values of spatiotemporal big data.

In addition, deep learning required for the soil gas monitoring and response system 100 to analyze soil gases is a machine learning technique using a deep neural network, and may analyze and predict the characteristics of an observed phenomenon.

More specifically, to quantitatively analyze environmental factors by using real-time environmental monitoring data, the soil gas monitoring and response system 100 may include a preprocessor 110, a dynamic characteristic processor 120, and a driving force processor 130.

First, according to one embodiment, the preprocessor 110 may configure a base dataset for observation data of soil gases and related environments.

The preprocessor 110 may configure and sort a data matrix according to the observation items and the observation time resolution of a complex environmental measurement dataset including soil gases. This sorted data matrix may be interpreted as a base dataset.

In addition, the preprocessor 110 may perform interpolation processing on missing data for each temporal domain resolution or temporal observation interval for the sorted base dataset. In addition, the preprocessor 110 may perform noise filtering on the data of an interpolated base dataset according to the purpose of use, and may also standardize and normalize the data.

Missing data may be interpreted as an element in which data corresponding to record does not exist or there is no data for record.

Next, according to one embodiment, the dynamic characteristic processor 120 may identify and extract the dynamic characteristics of the configured base dataset.

Specifically, the dynamic characteristic processor 120 may perform state-space modeling by generating a state-space model for each temporal domain resolution for a base dataset configured based on Wavelet analysis.

State space modeling may be interpreted as a preparatory process for multi-resolution analysis that may be measured in a time-frequency domain by performing Wavelet transformation on time series data based on soil gases.

Next, the dynamic characteristic processor 120 may select optimal state-space models according to the temporal domain resolution of the configured base dataset, and may select a potential driving force group among selected optimal state-space models.

The potential driving force group may be interpreted as an environmental cause responsible for change in a main target observation variable. In the present invention, the potential driving force group may be interpreted as a cause of changes in the concentration/flux of soil gases and their time-varying characteristics/spatiotemporal characteristics thereof.

Since it is difficult to determine a direct cause until cause analysis based on specific correlation is performed, a potential driving force group may be selected.

The dynamic characteristic processor 120 may extract the time-dependent variation characteristics of a selected potential driving force group, and may quantify the extracted variation characteristics as the dynamic characteristics of a main time-frequency domain through Wavelet analysis.

Next, according to one embodiment, the driving force processor 130 may identify and evaluate a driving force for soil gases based on the extracted dynamic characteristics.

Specifically, the driving force processor 130 may diagnose the multi-resolution correlation between the potential driving force of an optimal state-space model selected according to the temporal domain resolution of a configured base data set and observed data. In particular, the driving force processor 130 may diagnose correlation reflecting time delay and phase change between potential driving forces and observation data.

In addition, the driving force processor 130 may select a scale with the highest degree of relevance as the highest correlation scale in consideration of the degree of relevance between potential driving forces and observed data, based on the results of the performed correlation diagnosis.

Then, the driving force processor 130 may identify a driving force by using Wavelet energy ratio between potential driving forces and observation data and the correlation of the selected highest correlation scale. In addition, the driving force processor 130 may evaluate relative contribution by processing the linear combination between the cumulative energy ratio of the selected highest correlation scale and the explanatory force index of the state-space model.

This evaluated relative contribution may be used to determine what factors caused a sudden change in the base dataset. In addition, based on the identified and evaluated driving force, an optimal response scenario for the occurring phenomenon may be provided.

As a result, when the soil gas monitoring and response system 100 is used, the structural characteristics of various environmental factors that change according to time for various soil gases and environmental time series measurement data to be measured may be reflected. In addition, key environmental driving forces that cause change in observation values and the effects thereof may be systematically separated and identified using multi-resolution time-frequency domain analysis.

In an embodiment of the present invention, soil gases are monitored and key environmental driving forces that cause change in observation values are identified, but the present invention is not limited thereto. The present invention may be applied to analysis of real-time environmental data such as earthquakes and tsunamis, and based on the analysis data, it is possible to identify events that have occurred in the past, and to predict events that will occur in the future.

More specifically, the present invention may be applied to air pollution, PM2.5, VOCs, water resources, water quantity and quality investigation/evaluation/management, ecological environment, environmental impact assessment, geologic environmental impact assessment, climate change, real-time disaster response, earthquake research, and the like.

When the present invention is used, application processing such as prediction, geospatial application, and real-time response may be performed by using a process from pre-processing of a base dataset to identification of a driving force as a core process. For example, future climate change may be predicted using observation information from atmospheric gases or soil (forward forecasting). In addition, based on current climate observation data and related proxy data (isotopes, pollen data, tree rings, etc.) and observation information, climate change in the past geological period may be inferred (backward historical matching).

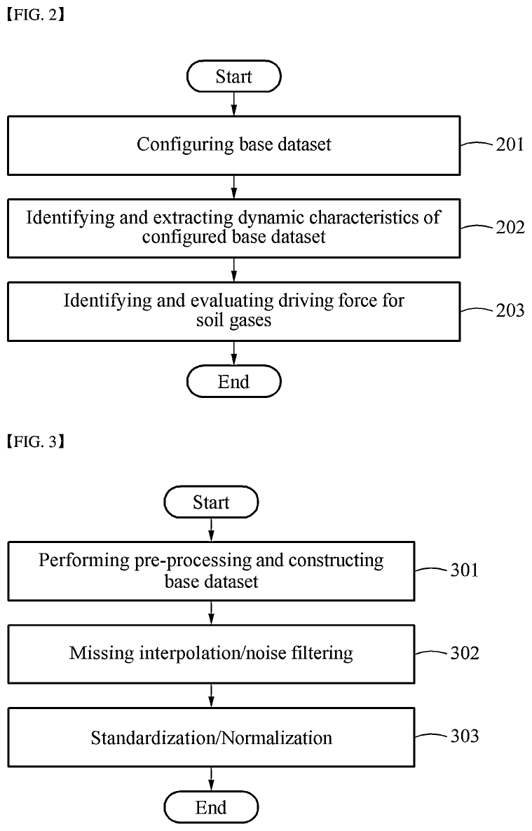

FIG. 2 is a flowchart for explaining a method of operating a soil gas monitoring and response system according to one embodiment.

According to one embodiment, the method of operating a soil gas monitoring and response system may include step 201 of configuring a base dataset for observation data of soil gases and related environments.

According to one embodiment, the base dataset may be monitored by dividing each numerical value monitored in real time according to a time series. For example, even when each value generated in carbon dioxide (CO₂) emitted from the soil has the same value, when the order of occurrence is different over time, the values may be interpreted as different meanings.

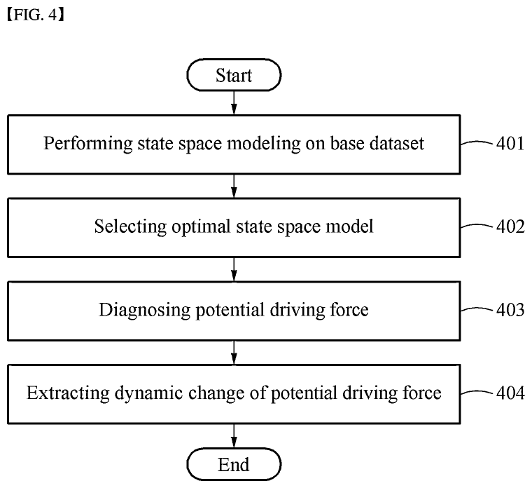

The process of constructing the base dataset may be described in more detail with reference to FIG. 3.

FIG. 3 is a flowchart for explaining a method of configuring a base dataset for observation data of soil gases and related environments.

According to one embodiment, the method of operating a soil gas monitoring and response system may include step 301 of performing pre-processing and constructing a base dataset to configure the base dataset for observation data of soil gases and related environments.

According to one embodiment, in the method of operating a soil gas monitoring and response system, a data matrix according to the observation items and the observation time resolution of a complex environmental measurement dataset including soil gases may be configured and sorted as a base dataset.

Observation are not perfect in the sorted base dataset. Accordingly, in the method of operating a soil gas monitoring and response system, insufficient observation data should be supplemented.

To supplement insufficient observation data, the method of operating a soil gas monitoring and response system may include step 302 of performing interpolation processing on missing data for each temporal domain resolution or temporal observation interval for the sorted base dataset.

Next, in the method of operating a soil gas monitoring and response system, noise filtering may be performed on the interpolated base dataset according to the purpose of analysis, and data may be standardized and normalized.

Referring back to FIG. 2, the method of operating a soil gas monitoring and response system may include step 202 of identifying and extracting the dynamic characteristics of the configured base dataset.

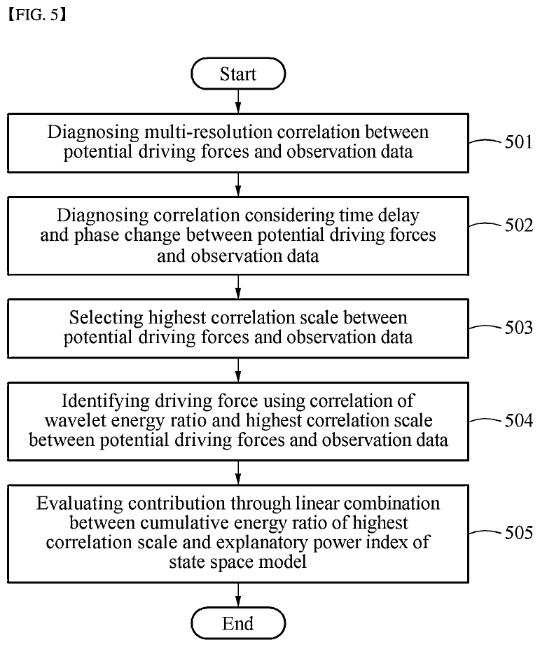

In the method of operating a soil gas monitoring and response system, the dynamic characteristics of a main time-frequency domain may be quantified, which will be described in detail with reference to FIG. 4.

FIG. 4 is a flowchart for explaining a process of identifying and extracting the dynamic characteristics of a configured base dataset.

The method of operating a soil gas monitoring and response system may include step 401 of generating a state space mode for a base dataset to identify and extract the dynamic characteristics of the base dataset.

Specifically, in the method of operating a soil gas monitoring and response system, state-space modeling may be performed on the configured base dataset for each temporal domain resolution.

For example, the state-space model may be interpreted as appropriately modeling the change state of a system inherent in the observed values of time series by classifying and modeling time series change in which each item and element constituting the base dataset appear into observation equations and state equations.

For example, in the present invention, it is possible to create a state-space model using various gases, surrounding environments, and climatic environments that may be factors of change in soil gases as items.

Next, the method of operating a soil gas monitoring and response system may include step 402 of selecting an optimal state-space model based on the temporal domain resolution of the configured base dataset.

Specifically, the number of optimal potential driving forces and the form of a residual covariance matrix may be selected based on model diagnostic indexes (ACI/AICc/BIC) and explanatory power (loading), and based on the above results, an optimal state-space model may be selected.

The residual covariance matrix may be interpreted as information expressing a covariance representing the relationship between the two values for which the difference is calculated for a residual, which is the difference between the most certain value obtained from observed or measured values and a calculated or theoretical value.

In addition, the method of operating a soil gas monitoring and response system may include step 403 of selecting and diagnosing the potential driving force group of the selected optimal state-space model and step 404 of extracting the dynamic change of the potential driving force.

For example, in the method of operating a soil gas monitoring and response system, by extracting the time-dependent variation characteristics of the selected potential driving force group and quantifying the dynamic characteristics of a main time-frequency domain through Wavelet analysis for the extracted variation characteristics, the dynamic characteristics of the base dataset may be identified and extracted. In this case, the variation characteristics may include at least one of the time-dependent dynamic characteristics/time-varying characteristics/spatial varying characteristics/spatiotemporal characteristics of the selected potential driving force group.

Referring back to FIG. 2, the method of operating a soil gas monitoring and response system may include step 203 of identifying and evaluating the driving force for soil gases based on the extracted dynamic characteristics. Based on this, the method of operating a soil gas monitoring and response system may provide an optimal response scenario based on the identified and evaluated driving force.

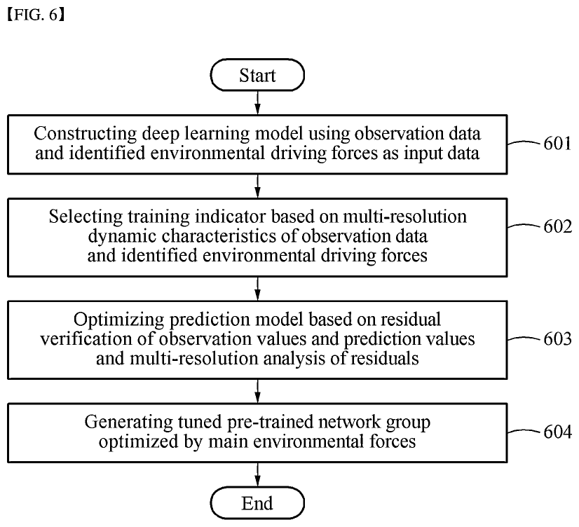

The process of identifying and evaluating the driving force for soil gases will be described in detail with reference to FIG. 5.

FIG. 5 is a flowchart for explaining a process of identifying and evaluating driving forces for soil gases based on extracted dynamic characteristics.

First, according to one embodiment, the method of operating a soil gas monitoring and response system may include step 501 of diagnosing multi-resolution correlation between potential driving forces and observation data to identify and evaluate the driving force for soil gases. In this case, correlation diagnosis considering time delay and phase change between potential driving forces and observation data may be performed (step 502).

In this specification, the process of diagnosing correlation is described separately in steps 501 and 502, but only at least one of steps 501 and 502 may be performed. For example, when only time delay and phase change are considered in diagnosing multi-resolution correlation, only step 502 may be performed. In addition, when only other information rather than time delay and phase change is considered in diagnosing multi-resolution correlation, only step 501 may be performed. In addition, in diagnosing multi-resolution correlation, when only time delay and phase change are considered in the last step of various processes, step 501 and step 502 may be performed sequentially.

Next, according to one embodiment, the method of operating a soil gas monitoring and response system may include step 503 of selecting a highest correlation scale between potential driving forces and observation data based on the results of performed correlation diagnosis.

That is, by evaluating correlation, the highest correlation scale related to the most correlated potential driving force and observed data may be selected.

Next, according to one embodiment, the method of operating a soil gas monitoring and response system may include step 504 of identifying a driving force using Wavelet energy ratio between potential driving forces and observation data and the correlation of the selected highest correlation scale.

In addition, according to one embodiment, the method of operating a soil gas monitoring and response system may include step 505 of evaluating contribution through a linear combination between the cumulative energy ratio of the highest correlation scale and the explanatory power index of the state-space model. For example, relative contribution may be evaluated by performing a linear combination between the cumulative energy ratio of the selected highest correlation scale and the explanatory power index of the state-space model.

According to one embodiment, in the method of operating a soil gas monitoring and response system, environmental factors may be identified and evaluated using a Wavelet-based multi-resolution state-space model SSM (MRSSM) approach.

Hereinafter, an MRSSM approach according to an embodiment will be described in more detail.

According to one embodiment, in the MRSSM approach, a state-space model (SSM) may be defined by Equations 1 and 2 below.

Xᵢ(t)=Σⱼ₌₁^{M}α_{i,j}Fᵢ(t)+μᵢ(t)+εᵢ(t) [Equation 1]

Fⱼ(t)=Fⱼ(t−1)+ηⱼ(t) [Equation 2]

Here, t denotes an observation time interval, M denotes potential driving force groups (PEDs), and Xᵢ(t), μᵢ(t), and εᵢ(t) denote an observation time series, a constant level parameter, and a specific error for an i-th observation variable (i is a real value), respectively.

In addition, Fⱼ(t) denotes j-th potential driving force (j is a real value) and includes seasonal or other cyclic trends, α_{i,j} denotes the factor loading of a j-th potential driving force group for i-th observation variable, εᵢ(t) denotes a residual term at time t, and εᵢ(t) and ηⱼ(t) denote values assumed to be white noise and evaluated through error covariance matrices R and Q.

According to one aspect, the SSM model (e.g., M and R, etc.) and the parameters of Equations 1 and 2 (e.g., α_{i,j} and εᵢ(t)) may be tested by combining a maximum likelihood method, Kalman filter/smoother, and an expectation-maximization (EM) algorithm.

In this case, in the structure of the error covariance matrix R, diagonal-and-equal, which assumes the same error covariance for all of time series, and diagonal-and-unequal, which assumes a specific error covariance for each of time series, may be considered. The potential driving force group M and the error covariance matrix R may be selected based on AICc (Akaike's Information Criterion corrected for small sample sizes).

According to one embodiment, in the MRSSM approach, a multi-resolution analysis part may be expressed using a Wavelet transform as shown in Equations 3 to 5 below.

f(t)=A_{J}(t)+Σₚ₌₁^{M}Dₚ [Equation 3]

Aₚ=Σ_{q=0}^{N-1}c_{p,q}ϕ_{p,q}(t) [Equation 4]

Dₚ=Σ_{q=0}^{N-1}d_{p,q}ψ_{p,q}(t) [Equation 5]

Here, f(t) denotes a time series of length N (N is a positive integer value), Aₚ and Dₚ denote the approximation and the detail value of the f(t) component at a decomposition level p, ϕ_{p,q}(t) and ψ_{p,q}(t) denote a scale function and a Wavelet mother function, respectively, and c_{p,q} and d_{p,q} denote an approximate coefficient which is a low frequency component at a decomposition level p and a time position q and a detail coefficient which is the high low frequency component of f(t), respectively.

In addition, the maximum decomposition level J may be determined by Equation 6 below.

$$J = ( \frac{log ( N / ( 2 {f}_{t} - 1 ) )}{log 2} ) \qquad [ Equation 6 ]$$

Here, fₗ denotes Wavelet filter length. In discrete Wavelet transform (DWT), decomposition level p and time position q may be sampled as powers of two for maximum decomposition level J and time series length N while continuous sampling is performed in continuous Wavelet transform (CWT).

In addition, the MRSSM approach according to one embodiment may detect maximum correlated time-frequency bands between times x and y using the maximum correlation coefficient rₘₐₓ defined in Equation 7 below.

rₘₐₓ=max[{|r^{d}_{xy}(p)|]_{p=1 to J},|r^{c}_{xy}|] [Equation 7]

Here, | | denotes an absolute value operator, r^{c}_{xy} denotes the scale-localized correlation coefficient of c_{J,q} for times x and y, and r^{d}_{xy}(p) denotes the scale-localized correlation coefficient of d_{p,q} for times x and y at decomposition level p. For example, when the value of the maximum decomposition level J is ‘5’, the decomposition level p may be expressed as (1, 2, 3, 4, 5).

In addition, in the case of r^{c}_{xy} and r^{d}_{xy}(p), the scale-localized correlation coefficients (i.e., r^{c}_{xy} and r^{d}_{xy}(p)) may be defined by Equation 8 below.

$${r}_{xy} = \frac{{C}_{xy} ( p )}{{σ}_{x} ( p ) {σ}_{y} ( p )} \qquad [ Equation 8 ]$$

Here, C_{xy} denotes the covariance between times x and y, and σₓ and σ_{y} denote the standard deviations of times x and y at the decomposition level p, respectively.

Equation 7 to 8 described above relate to scale-localized correlation analysis, may detect the maximum correlation frequency band between times x and y, and may be applied to find a time series with high correlation.

In addition, when the MRSSM approach according to one embodiment and Wavelet transformed coherency analysis (WTC) are used in combination, maximal correlated time-frequency bands between a potential driving force (PED) and observed data may be observed more clearly.

Here, WTC may represent time-frequency domain cross-correlation between an input time series x and an output time series y using CWT.

This may be based on the absolute value of cross-correlation (c_{xy}) using continuous Wavelet transform (CWT) when the time delay τ is included in Equation 8 above (i.e.,

$${r}_{xy} ( τ ) = \frac{{C}_{xy} ( p , τ )}{{σ}_{x} ( p , τ ) {σ}_{y} ( p , τ )} ) .$$

In this case, Wavelet coherence (i.e., time-frequency domain correlation) may be evaluated using a smoothing operator and Wavelet squared coherency (WSC), r_{xy}²(τ).

According to one aspect, the MRSSM approach according to one embodiment may evaluate the contribution of each potential driving force to target time series by using dynamic efficiency D_{ef}.

Here, the dynamic efficiency D_{ef} may be defined by Equation 9 below for calculating the factor loading α and the ratio Eₘₛ between accumulated Wavelet energy (E_{w}) and the total Wavelet energy in a main time-frequency band (scale).

D_{ef}=Eₘₛ×α [Equation 9]

Here, α represents the factor loading of a potential driving force for the time series of interest, and when the potential driving force affects a target time series, Eₘₛ may be evaluated using the Wavelet energy of the potential driving force.

So far, the technology for identifying and evaluating the driving force for soil gases through soil gas monitoring, which is the core technology of the present invention, has been described.

Hereinafter, applicable techniques such as prediction, geospatial application, and real-time response will be described based on the identified and evaluated driving force.

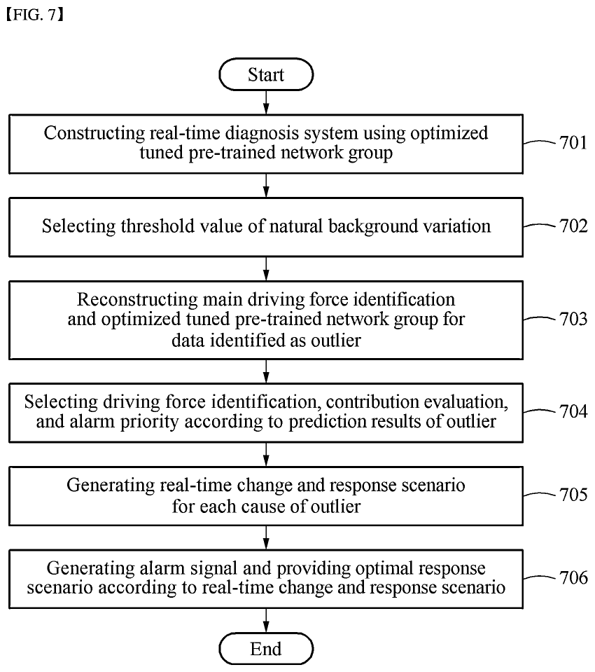

In particular, a response system using the results of soil gas monitoring will be described in detail with reference to FIGS. 6 to 8.

FIG. 6 is a flowchart for explaining a process of constructing a deep learning neural network model.

According to one embodiment, the method of operating a soil gas monitoring and response system may construct a deep neural network learning model using observation data of soil gases and related environments and environmental factors as input data at the same time.

According to the present invention, by intensively learning the key spatiotemporal characteristics of natural background variation of soil gases (CO₂, NO₂, CH₄, CO, C₂H₆, N₂O, SO₂, Rn) using the constructed deep neural network learning model, prediction of observation values and factors may be continuously improved. In addition, a real-time environmental monitoring, early warning, and response system may be implemented.

According to one embodiment, the method of operating a soil gas monitoring and response system may include step 601 of constructing a deep neural network model.

Specifically, a deep learning model using observation data of soil gases and related environments and an identified and evaluated driving force as input data may be constructed as the deep neural network model.

Next, according to one embodiment, the method of operating a soil gas monitoring and response system may include step 602 of selecting a training indicator based on the multi-resolution dynamic characteristics of observation data of soil gases and related environments and the identified and evaluated driving force and quantifying the training indicator. In addition, the method of operating a soil gas monitoring and response system may include step 603 of optimizing a prediction model based on the residual verification of observation data of soil gases and related environments and prediction values predicted from the deep neural network model and multi-resolution analysis of residuals.

Then, according to one embodiment, the method of operating a soil gas monitoring and response system may include step 604 of constructing a real-time environmental monitoring/early warning/response system by generating a tuned pre-trained network group through optimization processing by key environmental forces.

According to one embodiment, the method of operating a soil gas monitoring and response system may provide an optimal response scenario based on the identified and evaluated driving force by constructing a real-time response system using the constructed system.

FIG. 7 is a flowchart for explaining a process of constructing a real-time response system.

According to one embodiment, the method of operating a soil gas monitoring and response system may include step 701 of constructing a real-time diagnosis system using an optimized tuned pre-trained network group to construct a real-time response system. In addition, the method of operating a soil gas monitoring and response system may include step 702 of calculating the permissible range of natural background variation by selecting the threshold value of the natural background variation.

According to the method of operating a soil gas monitoring and response system, it may be judged whether the determined observation data exceeds the calculated permissible range. As a result of the judgment, main driving force identification and an optimized deep training network group for data determined as outliers may be reconstructed (step 703).

Next, the method of operating a soil gas monitoring and response system may include step 704 of identifying a driving force according to the prediction results of the outlier, evaluating relative contribution, and selecting alarm priority. In addition, the method of operating a soil gas monitoring and response system may include step 705 of generating a real-time change and response scenario for each cause of outliers and step 706 of generating an alarm signal according to generated a real-time change and response scenario and providing an optimal response scenario.

Accordingly, by configuring a deep neural network, a quantitative basis for the causality of a characteristic value and prediction for optimal learning may be present, and this may be applied in various ways.

In particular, according to the present invention, the key spatiotemporal change characteristics of target soil gases and environmental influencing factors may be intensively learned, and thus precise prediction may be efficiently performed.

In addition, real-time measurement data of various gaseous substances may be generally used without being limited to specific types of soil gases, and a leakage signal may be early detected by effectively separating/identifying natural background variation and anthropogenic leakage.

In addition, the present invention may contribute to evaluation of vulnerabilities and risks for target sites and environmental variables for specific environmental factors.

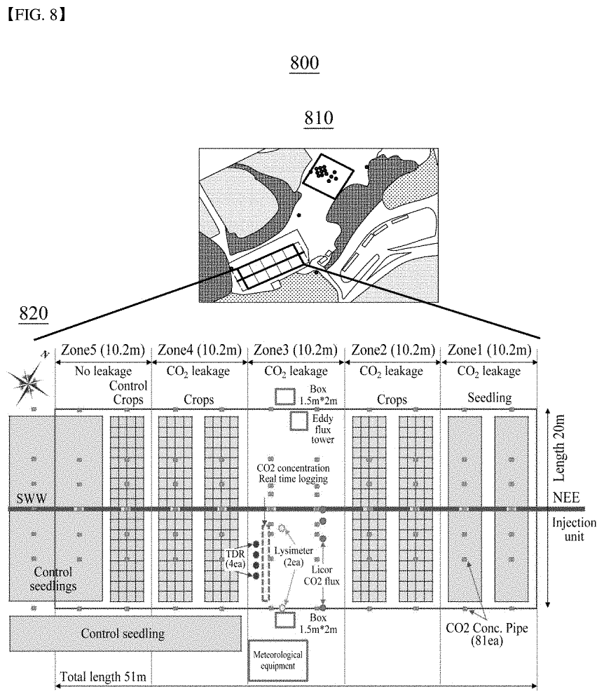

FIG. 8 shows a test site divided into zones for an experiment to observe emission of CO₂.

Drawing symbol 810 denotes photographic data describing the location and geographic features of a test site, and drawing symbol 820 denotes zones 1 to 5 partitioned for observation at the test site.

Specifically, drawing symbol 810 corresponds to an artificial CO₂ emission test site called environmental impact test facility (EIT) operated by a Korea-CO₂ storage environment monitoring and management (K-COSEM) Research Center to establish an integrated CO₂ storage environment management plan.

As shown by drawing symbol 820, CO₂ leakage is also measured in the remaining zones 2 to 5 except for zone 1.

In particular, the circle marked in zone 3 indicates an observation point for detecting carbon dioxide flux (FCO₂). Zone 3 may be used as a zone, i.e., zero-point data, for characterizing a baseline for CO₂ of the soil surface.

In the test site, a plurality of categories (four categories in the present embodiment) of real-time data may be collected for a certain period of time. Meteorological, atmospheric, soil characteristics, and soil respiration variables may be collected as observation data of soil gases and related environments, and a base data set may be constructed based on the data.

Meteorological variables (precipitation) such as rainfall (CR, mm) may be measured by an on-site automatic weather station at 10-minute intervals. In addition, atmospheric temperature, insolation, wind speed, relative humidity, and atmospheric pressure may be measured.

Soil properties such as soil temperature, soil volumetric moisture, and soil electrical conductivity may be measured by depth. For example, soil properties may be measured every 10 minutes.

In addition, soil respiration parameters (Rss) may be measured for surface water vapor content, CO₂ concentration, and flow rate measured using a closed automatic chamber. For example, soil respiration parameters may be measured every 30 minutes.

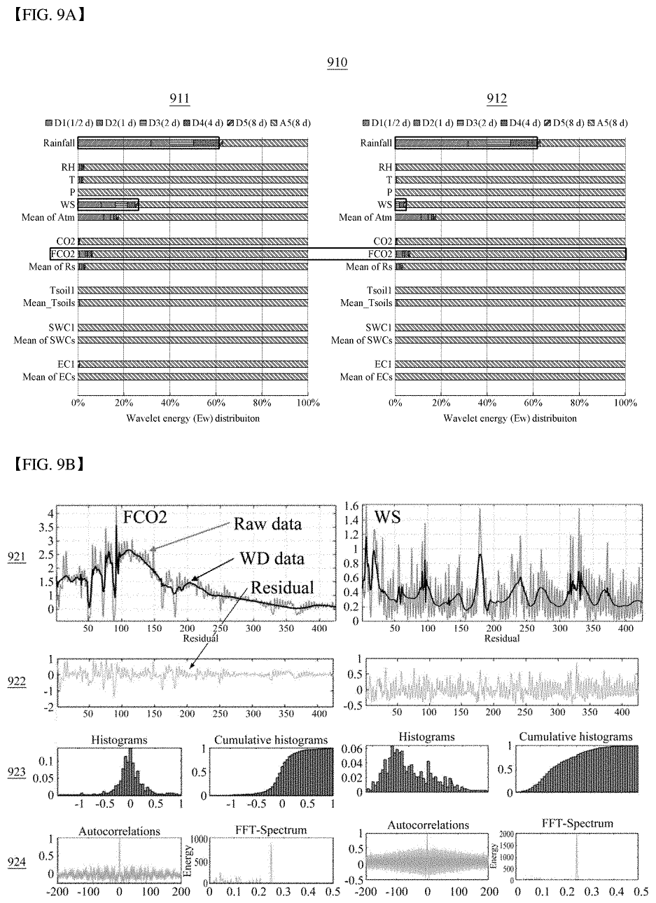

FIG. 9A shows a Wavelet energy distribution, and shows the Wavelet energy distribution of observation variables, Wavelet de-noising data, and potential environmental driving force groups by discrete Wavelet transform (DWT) for each frequency.

FIG. 9A shows Wavelet energy distribution for nine main observation variables.

For example, this embodiment shows energy distribution by discrete Wavelet transform (DWT) for Rainfall (accumulated rainfall), RH (relative humidity), T (atmospheric temperature), P (atmospheric pressure), WS (wind speed), CO₂ (CO₂ concentration), FCO₂ (CO₂ flux), Tsoil (soil temperature), SWC (soil moisture content), and EC (soil electrical conductivity), which are nine variables for soil gas observation.

Rainfall may be interpreted as rainfall information for an observation point. In addition, RH represents temperature-related relative humidity, T represents atmospheric temperature, P represents atmospheric pressure, WS represents wind speed, CO₂ represents carbon dioxide, and FCO₂ represents carbon dioxide flux at the soil surface.

FIG. 9A shows the results of discrete Wavelet transform (DWT) and Wavelet de-noising. Drawing symbol 911 corresponds to the distribution of Wavelet energy for each frequency based on discrete Wavelet for the raw data of major observation variables, and drawing symbol 912 represents Wavelet energy distribution information for the Wavelet de-noising data of major observation variables. In particular, D1 to D5 and A5 are Wavelet decomposition steps by discrete Wavelet analysis, and may represent the time-frequency scale of each component used for Wavelet analysis over a range of different lengths depending on the length of observation data.

In environmental time series for Wavelet energy distribution, a potential driving force group (PDF) may be decomposed into final approximate components (A5) and detailed components (D1 to D5) using discrete Wavelet transform DWT.

Among items constituting the equation of discrete Wavelet transform (DWT), Ap and Dp for the decomposition level may be included. Ap and Dp may apply a low frequency signal of 0.25 cycles or less and a high frequency signal of 0.25 to 0.5 cycles at each decomposition level (p). When considering a decomposition level according to the length of observation data, the maximum decomposition level A5 according to an embodiment corresponds to a scale of 25*6 time (i.e., 8 days), which is variously selected according to the observation interval and length of raw data.

Then, according to one embodiment, all time-frequency scales for decomposition levels may be ½ day (D1), 1 day (D2), 2 days (D3), 4 days (D4), and 8 days (D5).

For example, processes from D1 to D3 may be considered short-term (scale of 2 days), and processes of D5 and A5 may be considered long-term (scale of 8 days or more) or seasonal.

As shown in drawing symbols 911 and 912, rainfall shows a distribution of 60% or more from D1 to D4, whereas WS shows a distribution of just over 20%. In addition, CO₂ shows a major Wavelet energy (Ew) distribution of a short-term scale, whereas FCO₂ shows a major Ew distribution of a relatively long-term from D5 to A5.

In addition, Tsoils and SWCs showed a moderate change in Ew according to soil depth, but EC showed a sharp change in Ew in the most severe soil (EC3).

FIG. 9B includes graphs 920 showing major observation data and the noise filtering effects thereof.

First, drawing symbol 921 is a graph showing carbon dioxide flux (FCO₂) and change in wind speed (WS). Raw data represents measured data and Wavelet denoise (WD) data represents a signal obtained by removing noise from raw data.

In addition, drawing symbol 922 corresponds to residual data for raw data and WD data. Residual data is useful for the interpretation of rapidly changing observation data, and may be used to identify, evaluate, and predict driving forces hidden in a short frequency domain. For example, residual data may be used for analysis and prediction of earthquake data.

In addition, drawing symbols 923 and 924 indicate various analysis values for collected raw data, for example, a histogram, a cumulative histogram, an autocorrelation diagram, an FFT spectrum, and the like for raw data.

According to the present invention, data observed in various environments may be reflected by using the data of FIGS. 9A and 9B.

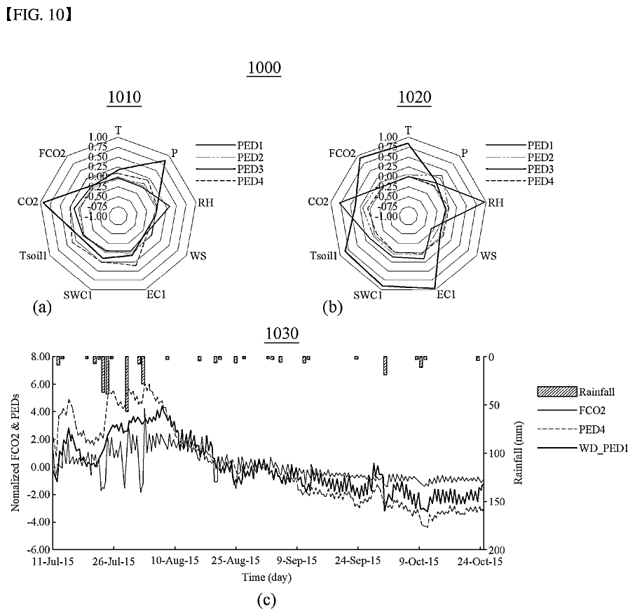

FIG. 10 includes diagrams showing potential environmental driving force groups (Potential EDs) extracted by state-space modeling.

FIG. 10 includes diagrams showing a dynamic factor (DyF) based on raw data and a dynamic factor (DyF) based on Wavelet de-noising (WD).

Thereamong, drawing symbol 1010 shows dynamic factor loading for raw data, and drawing symbol 1020 shows dynamic factor loading for Wavelet de-noise (WD) data.

For example, by selecting PED4 of a raw data model or WD_PED1 of a Wavelet de-noising data model as a main potential driving force group for Soil CO₂ flux (FCO₂), which is a target variable for evaluation in one embodiment, for each element, the degree of element load and change over time may be confirmed.

At drawing symbol 1030, Wavelet energy distribution for each frequency domain may be confirmed as compared to the raw data of carbon dioxide flux over time and main environmental time series observation data and Wavelet de-noised data.

For example, when a user wants to know the effect of change in soil gas flux and the environmental driving force when it rains, by increasing learning intensity for raw data and a signal (PED4) corresponding to the potential environmental driving force of the raw data, the change characteristics of a target variable due to the influence of environmental factors may be analyzed/learned (trained) in detail. When a user wants to know the effect of change in soil gas flux and the environmental driving force when it is not raining, by performing analysis and learning on a noise-removed signal (WD_PED1), applicability may be increased according to an analysis target, time, and purpose.

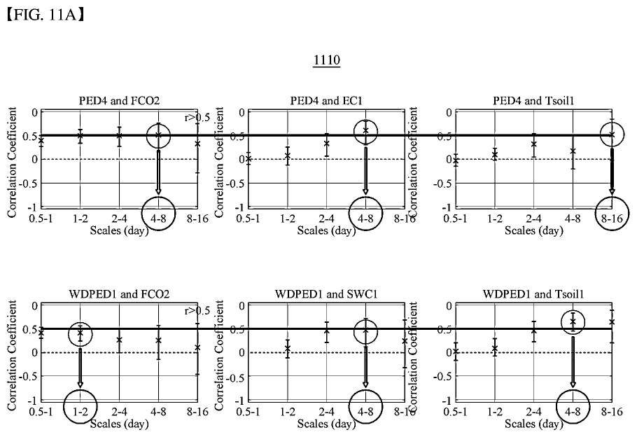

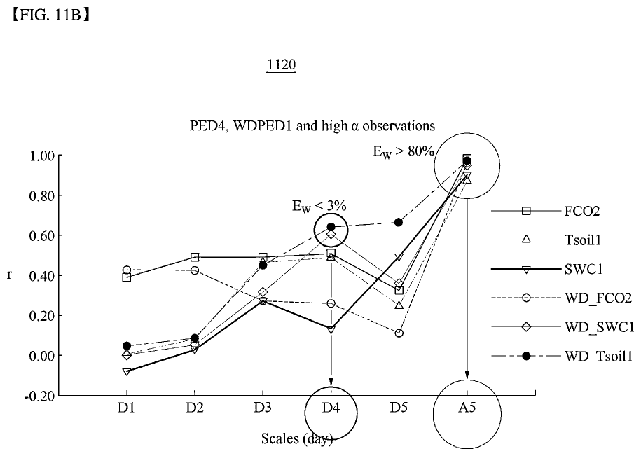

In FIG. 11A, as shown in drawing symbol 1110, to determine which frequency among observation data is a core frequency, a scale-versus-correlation diagram may be considered.

It may be determined which frequency is a core frequency by checking a correlation coefficient (r) against a scale for each element constituting observation data. For example, when a correlation coefficient (r) for each discrete Wavelet decomposition level is 0.5 or more, PED4 and FCO₂ show high correlation coefficients on a scale of 4 to 8 days. In addition, noise-removed PED1 and FCO₂ shows high correlation coefficients on a scale of 1 to 2 days.

FIG. 11B shows a scale-versus-correlation diagram 1120.

In one embodiment, a high correlation is observed in D4 and A5. To confirm which element is an important element, Wavelet energy distribution (Ew) at the corresponding scale may be checked.

As shown in correlation 1120, Wavelet energy distribution on the scale of D4 is less than 3%, and Wavelet energy distribution on the scale of A5 is more than 80%. It can be confirmed that the maximum correlation is observed at A5, that is, the scale after 8 days. These measurements suggest that main environmental driving forces (MEDs) for FCO₂ are closely related to an environmental process lasting more than 8 days. Thus, it is possible to confirm a frequency band in which the influence of a relevant main environmental driving force is greatest. That is, it can be confirmed which element is a main driving force for change in soil gases among observation data.

When a user wants to check information on detailed elements rather than energy distribution, it is possible to analyze detailed elements by converting the signal of a specific frequency band of interest into a time domain and confirming the time domain.

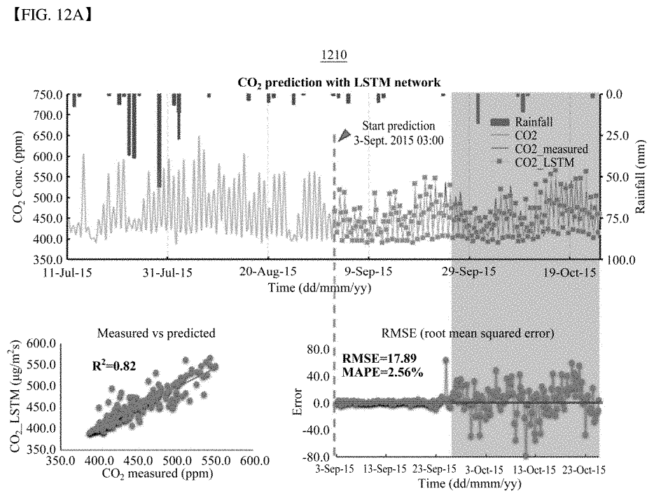

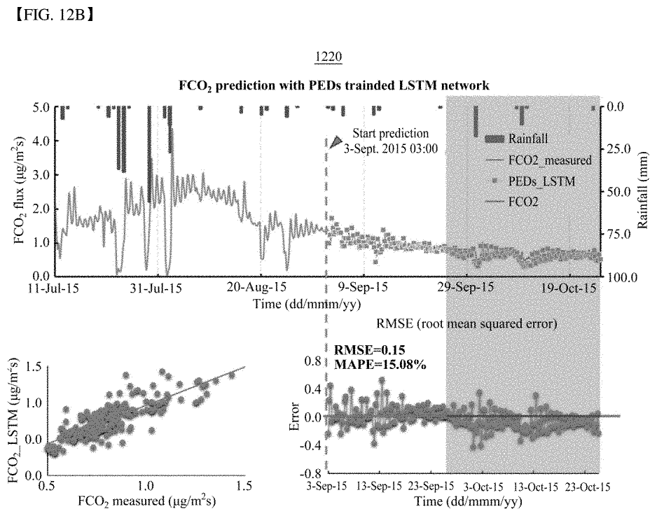

FIG. 12A shows an example of deep learning prediction using only raw observation data as input data.

Drawing symbol 1210 shows the results of real-time observation of CO₂ concentration at an observation point and the results of performing deep learning based on the observation results. At drawing symbol 1210, points shown in each graph correspond to predicted values, and each predicted value is indicated by a correlation with respect to a scale together with raw data.

For example, error rate with respect to time shows a sharp increase after September 15th. This deep learning results show a sudden increase in residuals.

The reason that error rate for residuals increases is that there is accumulation of errors or a training data set used for training does not properly reflect the changed characteristics of a prediction period.

FIGS. 12A and 12B show the results of deep learning using summer observation data, and show that error significantly increases from a specific time when fitting autumn observation data.

In other words, the error rate for the predicted values of CO₂ concentration and flux (FCO₂) increases remarkably after September 15th. This indicates that a series of environmental changes that deviate from the prediction results of a deep neural network trained with training data reflecting the change characteristics of summer occurred from the time when the error significantly increased. Thus, it can be seen that the cause of the change in the error rate is environmental change according to season rather than change due to accumulation of errors.

Accordingly, as in one embodiment, the present invention may also be used to detect the structural change points of environmental factors through analysis of increase in cumulative error.

In FIG. 12B, as compared to FIG. 12A, it can be confirmed that as a result of strengthening learning for key features by using not only the observed value of FCO₂ but also the previously identified environmental driving force as training data, efficiency against errors is increased. That is, it can be confirmed that efficiency against errors is much improved through deep learning, which reinforces learning about the dynamic characteristics of key environmental driving forces identified by using observation data and environmental driving forces at the same time to predict a target variable.

In addition, as a result of evaluating and predicting main environmental driving forces controlling the natural background variation (baseline) of the CO₂ flux (FCO₂) of the soil surface at a CO₂ test site (EIT), it can be confirmed that the Wavelet de-noising method (WD) is effective in improving the performance of the state space model (SSM), especially computation time and element loading.

In addition, deep learning may be used to predict complex environmental processes, and furthermore, deep learning may be used to construct a real-time CO₂ leakage monitoring system. Even for nonstationary environmental observation data, the core Wavelet-based multi-resolution SSM (MRSSM) approach may identify and evaluate environmental factors behind complex physicochemical, biological, and ecological processes in both time and space.

The apparatus described above may be implemented as a hardware component, a software component, and/or a combination of hardware components and software components. For example, the apparatus and components described in the embodiments may be achieved using one or more general purpose or special purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and one or more software applications executing on the operating system. In addition, the processing device may access, store, manipulate, process, and generate data in response to execution of the software. For ease of understanding, the processing apparatus may be described as being used singly, but those skilled in the art will recognize that the processing apparatus may include a plurality of processing elements and/or a plurality of types of processing elements. For example, the processing apparatus may include a plurality of processors or one processor and one controller. Other processing configurations, such as a parallel processor, are also possible.

The software may include computer programs, code, instructions, or a combination of one or more of the foregoing, configure the processing apparatus to operate as desired, or command the processing apparatus, either independently or collectively. In order to be interpreted by a processing device or to provide instructions or data to a processing device, the software and/or data may be embodied permanently or temporarily in any type of a machine, a component, a physical device, a virtual device, a computer storage medium or device, or a transmission signal wave. The software may be distributed over a networked computer system and stored or executed in a distributed manner. The software and data may be stored in one or more computer-readable recording media.

The methods according to the embodiments of the present invention may be implemented in the form of a program command that can be executed through various computer means and recorded in a computer-readable medium. The computer-readable medium can store program commands, data files, data structures or combinations thereof. The program commands recorded in the medium may be specially designed and configured for the present invention or be known to those skilled in the field of computer software. Examples of a computer-readable recording medium include magnetic media such as hard disks, floppy disks and magnetic tapes, optical media such as CD-ROMs and DVDs, magneto-optical media such as floptical disks, or hardware devices such as ROMs, RAMs and flash memories, which are specially configured to store and execute program commands Examples of the program commands include machine language code created by a compiler and high-level language code executable by a computer using an interpreter and the like. The hardware devices described above may be configured to operate as one or more software modules to perform the operations of the embodiments, and vice versa.

Although the present invention has been described with reference to limited embodiments and drawings, it should be understood by those skilled in the art that various changes and modifications may be made therein. For example, the described techniques may be performed in a different order than the described methods, and/or components of the described systems, structures, devices, circuits, etc., may be combined in a manner that is different from the described method, or appropriate results may be achieved even if replaced by other components or equivalents.

Therefore, other embodiments, other examples, and equivalents to the claims are within the scope of the following claims.

1. A method of operating a soil gas monitoring and response system, comprising:
a step of configuring a base dataset for observation data of soil gases and related environments;
a step of identifying and extracting dynamic characteristics of the configured base dataset; and
a step of identifying and evaluating a driving force for soil gases based on the extracted dynamic characteristics,
wherein an optimal response scenario is provided based on the identified and evaluated driving force.

2. The method according to claim 1, wherein the step of configuring a base dataset for observation data of soil gases and related environments comprises a step of configuring and aligning, as the base dataset, a data matrix according to observation items and observation time resolution of a complex environmental measurement dataset comprising the soil gases;
a step of performing interpolation processing on missing data for each temporal domain resolution or temporal observation interval for the sorted base dataset; and
a step of performing noise filtering on data of the interpolated base dataset, and standardizing and normalizing the noise-filtered results.

3. The method according to claim 1, wherein the step of identifying and extracting dynamic characteristics of the configured base dataset comprises a step of performing state-space modeling on the configured base dataset for each temporal domain resolution;
a step of selecting an optimal state-space model according to the temporal domain resolution of the configured base dataset;
a step of selecting a potential driving force group of the selected optimal state-space model;
a step of extracting time-dependent variation characteristics of the selected potential driving force group; and
a step of quantifying dynamic characteristics of a main time-frequency domain through Wavelet analysis for the extracted variation characteristics,
wherein the variation characteristics comprise at least one of time-dependent dynamic characteristics, time-varying characteristics, spatial characteristics, and spatiotemporal characteristics of the selected potential driving force group.

4. The method according to claim 3, wherein the step of selecting an optimal state-space model comprises a step of selecting the number of optimal potential driving forces and a form of a residual covariance matrix based on model diagnostic indexes (AIC, AICc, BIC) and explanatory power (loading).

5. The method according to claim 1, wherein the step of identifying and evaluating driving forces for soil gases based on the extracted dynamic characteristics comprises a step of diagnosing multi-resolution correlation between observation data and potential driving forces of an optimal state-space model selected according to a temporal domain resolution of the configured base dataset, and performing correlation diagnosis reflecting time delay and phase change between the potential driving forces and the observation data;
a step of selecting a highest correlation scale between the potential driving forces and the observation data based on results of the performed correlation diagnosis;
a step of identifying a driving force using a Wavelet energy ratio between the potential driving forces and the observation data and a correlation of the selected highest correlation scale; and
a step of evaluating relative contribution by processing a linear combination between a cumulative energy ratio of the selected highest correlation scale and an explanatory power index of the state-space model.

6. The method according to claim 1, further comprising a step of constructing a deep learning model for real-time diagnosis of the driving force.

7. The method according to claim 6, wherein the step of constructing a deep learning model comprises a step of constructing, as a deep neural network model, a deep learning model using the observation data of soil gases and related environments and the identified and evaluated driving force as input data;
a step of quantifying a training indicator by selecting the training indicator based on multi-resolution dynamic characteristics of the observation data of soil gases and related environments and the identified and evaluated driving force;
a step of optimizing a prediction model based on residual verification of observation values measured from the observation data of soil gases and related environments and prediction values predicted from the deep neural network model and multi-resolution analysis of residuals; and
a step of generating a tuned pre-trained network group by performing optimization processing by main environmental forces.

8. The method according to claim 6, further comprising a step of constructing a real-time response system for providing the optimal response scenario.

9. The method according to claim 8, wherein the step of constructing a real-time response system comprises a step of constructing a real-time diagnosis system using the generated tuned pre-trained network group;
a step of calculating a permissible range of a natural background variation by selecting a threshold value of the natural background variation;
a step of re-identifying a driving force for data determined as an outlier with respect to the threshold value, and reconfiguring an optimized deep training network group based on the re-identified driving force;
a step of identifying a driving force according to prediction results of the outlier, evaluating relative contribution, and selecting an alarm priority;
a step of generating a real-time change and response scenario for each cause of the outlier; and
a step of generating an alarm signal according to the generated real-time change and response scenario, and providing an optimal response scenario.

10. A soil gas monitoring and response system, comprising:
a preprocessor for configuring a base dataset for observation data of soil gases and related environments;
a dynamic characteristic processor for identifying and extracting dynamic characteristics of the configured base dataset; and
a driving force processor for identifying and evaluating a driving force for soil gases based on the extracted dynamic characteristics,
wherein an optimal response scenario is provided based on the identified and evaluated driving force.

11. The soil gas monitoring and response system according to claim 10, wherein the preprocessor configures and aligns, as the base dataset, a data matrix according to observation items and observation time resolution of a complex environmental measurement dataset comprising the soil gases; performs interpolation processing on missing data for each temporal domain resolution or temporal observation interval for the sorted base dataset; and performs noise filtering on data of the interpolated base dataset, and standardizes and normalizes the noise-filtered results.

12. The soil gas monitoring and response system according to claim 10, wherein the dynamic characteristic processor performs state-space modeling on the configured base dataset for each temporal domain resolution; selects an optimal state-space model according to the temporal domain resolution of the configured base dataset; selects a potential driving force group of the selected optimal state-space model; extracts time-dependent variation characteristics of the selected potential driving force group; and quantifies dynamic characteristics of a main time-frequency domain through Wavelet analysis for the extracted variation characteristics.

13. The soil gas monitoring and response system according to claim 10, wherein the driving force processor diagnoses multi-resolution correlation between observation data and potential driving forces of an optimal state-space model selected according to a temporal domain resolution of the configured base dataset, and performs correlation diagnosis reflecting time delay and phase change between the potential driving forces and the observation data; selects a highest correlation scale between the potential driving forces and the observation data based on results of the performed correlation diagnosis; identifies a driving force using a Wavelet energy ratio between the potential driving forces and the observation data and a correlation of the selected highest correlation scale; and evaluates relative contribution by processing a linear combination between a cumulative energy ratio of the selected highest correlation scale and an explanatory power index of the state-space model.
